(19) United States
(12) Patent Application Publication
(10) Pub. No.: US 2024/0165333 A1
(43) Pub. Date: May 23, 2024

(54) PARENTERAL INJECTION SYSTEM

(71) Applicant: Action Medical Technologies, LLC, Conshohocken, PA (US)

(72) Inventors: Joseph B. Hoffer, Newport, PA (US); Mark W. Pursel, Grantville, PA (US)

(73) Assignee: Action Medical Technologies, LLC, Conshohocken, PA (US)

(21) Appl. No.: 18/514,961

(22) Filed: Nov. 20, 2023

Related U.S. Application Data
(60) Provisional application No. 63/384,486, filed on Nov. 21, 2022.

(51) Int. Cl. A61M 5/24; A61M 5/20; A61M 5/32

(52) U.S. Cl. CPC A61M 5/24; A61M 5/20; A61M 5/326; A61M 2005/206; A61M 2005/2403

(57) ABSTRACT

A parenteral injection system includes a sled including a sleeve and configured to be in contact with a patient's skin; and an enclosure configured to secure an injector having a needle and be fed into the sleeve.

### CROSS REFERENCE TO RELATED APPLICATION

This application claims the benefit of U.S. Provisional Patent Application No. 63/384,486, filed Nov. 21, 2022, which is incorporated by reference for all purposes as if fully set forth herein.

### TECHNICAL FIELD

The present disclosure relates generally to injectors for medicaments and other fluids, and more particularly, to a parenteral injection system that guides a needle of an injector into a target site.

### BACKGROUND

An intradermal parernal injection systems puts medication within the skin organ. This medicament is injected into the epidermis or the dermis. For this intradermal injection, the administrator may stretch the skin of the patient taut, insert the needle into the patient at an angle of 5°-15°, and administer the medicament or other fluid into the skin to case formation of a weal or bleb to appear in the patient's skin, which indicates that the medicament has been injected in the dermis. However, seeing the weal or bleb is dependent on the administrator inserting the needle at the correct angle. If the administrator mistakenly inserts the needle at the incorrect angle, the administrator may place the medicament incorrectly or may have to reinsert the needle to attempt another injection.

### SUMMARY

According to embodiments, an injection system can provide predrawn or prefilled medication in an injector contained in a safety tray/enclosure. While in the safety tray/enclosure, the medication and injector are protected from contamination and damage so that they can be safely stored and transported. Features can be included to orient the bevel of a needle, align the injector, and set the maximum needle depth for the medication administration. It is expected that after use the entire injection system will be discarded.

According to an embodiment of the present disclosure, a parenteral injection system includes a sled including a sleeve and configured to be in contact with a patient's skin; and an enclosure configured to secure an injector having a needle and be fed into the sleeve.

In an aspect, the enclosure is configured to hold or store the injector that has been occupied with a medicament.

In an aspect, the injector is a syringe.

In an aspect, the enclosure in one of a kit of enclosures and is color coded.

In an aspect, the sled and/or the enclosure glows in the dark.

In an aspect, the sleeve is at a predetermined angle with respect to the patient's skin while the sled is in contact with the patient's skin.

In an aspect, an angle of the needle and an insertion depth of the needle corresponds to the predetermined angle of the sleeve.

In an aspect, the enclosure is a substantially cubic box that includes a body, a cover, and a needle protector.

In an aspect, the cover includes a locking feature to secure the cover to the body when the enclosure is closed.

In an aspect, the cover includes a clear portion in which to view the injector while inside the enclosure.

In an aspect, the needle protector is rotatably attached to the body and covers the needle while the injector is secured in the enclosure.

In an aspect, the body is keyed with the injector such that the needle must be in a predetermined orientation for the injector to be secured in the enclosure.

In an aspect, the sleeve includes a keying feature such that the enclosure must be in a predetermined orientation to be inserted into the sleeve.

According to an embodiment of the present disclosure, a method of parenteral injection includes securing an injector into an enclosure including a needle protector that covers a needle of the injector and is rotatably attached to the enclosure, the injector including a medicament; pressing a sled including a sleeve on a surface of a patient's skin; feeding the enclosure into the sleeve until the needle protector rotates to expose the needle; puncturing the patient's skin with the needle; and delivering the medication into the patient's skin.

In an aspect, the method can further include causing the needle protector to rotate and cover the needle while withdrawing the enclosure from the sleeve.

In an aspect, an operator presses the sled with one hand and feeds the enclosure, punctures the patient's skin, and delivers the medication with a second hand.

In an aspect, pressing the sled on the patient's skin makes the patient's skin taut.

In an aspect, the sleeve includes a keying feature such that the enclosure must be in a predetermined orientation to be inserted into the sleeve.

In an aspect, the enclosure is keyed with the injector such that the needle must be in a predetermined orientation for the injector to be secured in the enclosure.

In an aspect, an angle and depth of the needle with respect to the surface of the patient's skin is predetermined by a configuration of the needle within the enclosure and the enclosure within the sleeve.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### BRIEF DESCRIPTION OF THE DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1 is a side view of a parenteral injection apparatus according to an embodiment of the present disclosure.

FIG. 2 is perspective view of an enclosure and an injector of a parenteral injection apparatus.

FIG. 3 is a perspective view of an injector in an enclosure.

FIG. 4 is a perspective view of an injector in an enclosure.

FIG. 5 is an enlarged partial perspective view of injector in an enclosure.

FIG. 6 is a perspective view of a parenteral injection system in a preparation position.

FIG. 7 is side view of the parenteral injection system in a preparation position.

FIG. 8 is a partial perspective view of a parenteral injection system in an intermediate position.

FIG. 9 is a side view of a parenteral injection system in an injection position.

FIG. 10 is a partial perspective view of a parenteral injection system in an injection position.

FIG. 11 is a partial perspective view of the sled 130.

FIG. 12 is a partial perspective view of a parenteral injection system while an enclosure with injector is being withdrawn.

FIG. 13 is a partial perspective rear view of an enclosure.

FIG. 14 is a perspective view of an injector.

### DETAILED DESCRIPTION

The following discussion omits or only briefly describes conventional features of injectors that are apparent to those skilled in the art. It is noted that various embodiments are described in detail with reference to the drawings, in which like reference numerals represent like parts and assemblies throughout the several views. Reference to various embodiments does not limit the scope of the claims attached hereto. Additionally, any examples set forth in this specification are intended to be non-limiting and merely set forth some of the many possible embodiments for the appended claims. Further, particular features described herein can be used in combination with other described features in each of the various possible combinations and permutations.

Unless otherwise specifically defined herein, all terms are to be given their broadest reasonable interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “includes” and/or “including,” when used in this specification, specify the presence of stated features, elements, and/or components, but do not preclude the presence or addition of one or more other features, steps, operations, elements, components, and/or groups thereof. In the description, relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively or operably connected” is such an attachment, coupling or connection that allows the pertinent structures to operate as intended by virtue of that relationship. The terms “medicament” or “fluid” as used herein refers to any substance for delivery to a target. For example, these terms include anticoagulants, vaccines, biologics, or any other injectable fluids.

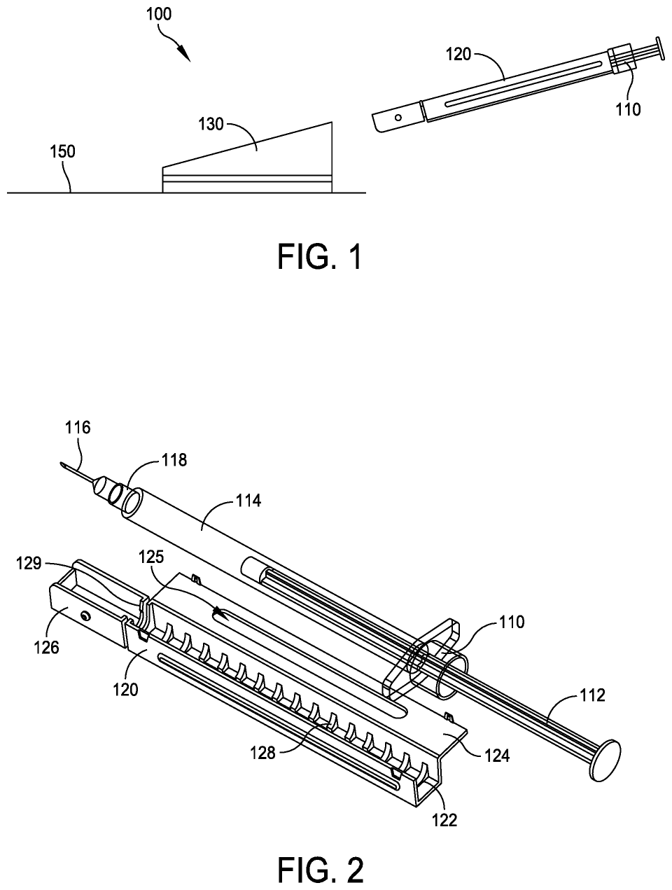

Embodiments of the present disclosure relate generally to a parenteral injection system and method to inject medicaments and other fluids. More particularly, embodiments guide a needle of an injector into a target site. Embodiments of the parenteral injection system are described below with reference to FIGS. 1-14.

FIG. 1 is a side view showing components of an example parenteral injection system 100 that can include an injector 110, a tray or an enclosure 120 to house the injector 110, and a sled 130 used to guide the enclosure 120 containing the injector 110 to a patient's skin 150. As shown, FIG. 1 illustrates that the sled 130 can be placed on a surface of a patient's skin 150. As further described below, the sled 130 can include a guide or sleeve 132 in which the enclosure 120 can be inserted and directed to the patient's skin 150.

FIGS. 2-5 provide different views of the injector 110 and the enclosure 120. The enclosure is used to safely house the injector 110 for transport and storage prior to being used for medicament delivery. Medicament can be drawn into the injector 110 and the prepared injector 110 can be placed into the enclosure 120. As shown, the injector 110 can be a syringe. As is well known, the syringe 110 can include a plunger 112 with a pressing flange at one end and a seal on the other end, a barrel 114 configured to contain a liquid medicament, a needle 116, and a hub 118 to which the needle 116 is attached, as indicated in FIG. 2. The enclosure 120 can be sized and structured to match a certain type or size of injector 110. A kit of enclosures 120 can be provided having different enclosures 120 configured to mate with different injectors 110. In an aspect, enclosures 120 can be marked or color coded as an indication of its size and/or features. In an aspect, the enclosure 120 and/or sled 130 can glow in the dark. In an aspect, the enclosure can include haptic sensing features such that an administrator can determine different enclosures by touch or feel.

An end of the needle 116 and the gauge size of the needle 116 can correspond to the type of injection being administered. For example, for the cases in which a needle 116 is being injected intradermally, the needle 116 can have a so-called intradermal-beveled end and can be a 26 or 27-gauge needle, or any suitable gauge. In one or more cases, the length of the needle 116 and the insertion depth of the needle 116 can correspond to the angle of the sleeve 132 and the location of syringe within the enclosure. Because this is an intradermal injection, the intradermal grind bevel of the needle 116 must be oriented in a certain direction and set for a specific depth of penetration of the needle 116; this must be tightly controlled. For example, for optimal results in an intradermal injection, the needle bevel should be facing up. The enclosure 120 and mating syringe 110 are designed with keying features (described below) to ensure proper orientation of the needle bevel and control the needle depth prior to medicament administration.

The enclosure 120 can be a substantially rigid cubic box that can include a body 122 having an interior cavity, a cover 124, and a needle protector 126. In an aspect, the enclosure 120 is plastic. As shown in FIG. 2, the interior cavity of the body 122 can include ribs 128 or structures used to secure the substantially round barrel 114. Additionally, the geometry of the ribs 128 can set the longitudinal and lateral location of the injector 110 within the enclosure 120. In turn, the geometry of the ribs 128 and orientation of the keying features will set the location and tip of the needle 116 within the enclosure 120, which is a parameter necessary to determine the needle depth during medicament administration. The cover 124 can be substantially rectangular and rotatably and/or hingedly attached to the body 122 to open access to the interior cavity and closed. The enclosure 120 can include locking tabs or another mechanism to secure the cover 124 when closed. As shown, the cover 124 can also include an opening, clear area, or slot 125 used to view the injector 110 once the injector 110 is placed into the enclosure 120 and the cover 124 is closed. In an aspect, the material of the cover 124 is transparent or semi-opaque to permit a user to see through the cover 124.

The needle protector 126 protects the needle 116 from most sides. As shown, the needle protector 126 can be substantially cubic shaped with one side open or omitted to allow the injector 110 be inserted and the needle 116 projected towards the patient's skin, as described below. The needle protector 126 can also be rotatably or hingedly attached to the body 122, as described below.

FIG. 2 also shows that the needle protector 126 can include a detent 129. The detent 129 is a structural feature that can secure the hub 118 of the injector 110 into the enclosure 120. The detent 129 can be defined as a substantially hemispheric opening or cutout of a rib within or an end wall of the needle protector 126. The geometry of the cutout of the detent 129 can be sized slightly smaller than a diameter of the hub 118 such that the hub 118 can be press fit into the cutout and the detent 129 will secure the hub 118, and with it, the injector 110 into place within the enclosure 120. In an aspect, rather than being completely rounded, the hub 118 can include a straight flat surface. In an aspect, the hub 118 or the barrel 114 can include a protrusion or raised rib 111, as shown in FIG. 14. The flat surface on the hub 118 or the raised rib 111 can be used as a key or indicator as to which direction the needle bevel must be oriented. Correspondingly, the cutout of the detent 129 can include mating geometry so that the hub 118 can fit into the detent 129 in only one orientation. The orientation of the hub 118 within the detent 129 can then determine the orientation of the needle bevel within the enclosure 120. For example, the needle bevel must be oriented in the direction in which the injector 110 is placed into the enclosure 120, i.e., away from the missing side of the needle protector shown in FIG. 2.

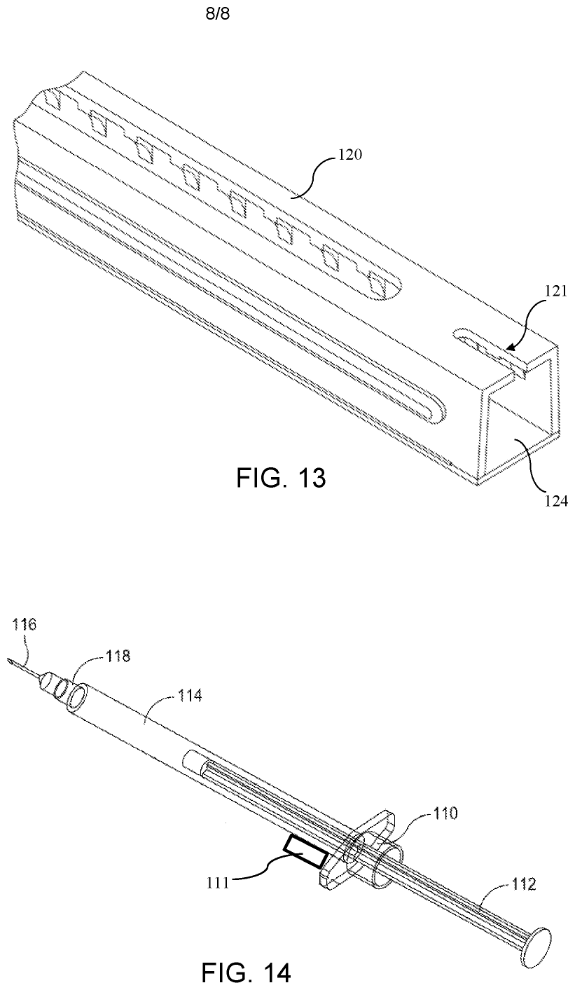

In an aspect, the enclosure 120 can include a recess, depression, or slot 121 on a rear side opposite to the cover 124, like that shown in FIG. 13, in which to mate with the raised rib 111 on the syringe 110, shown in FIG. 14 to key the orientation of the syringe 110 in the enclosure 120. The length of the slot 121 and the length of the raised rib and location of the raised rib 110 on the syringe 110 can be used to set the location of the needle 116 in the parenteral injection system 100. With this configuration, the syringe 110 must be oriented such that the raised rib 111 on the syringe 110 fits into the slot 121 of the enclosure 120 for the cover 124 of the enclosure to be closed and locked with the syringe 110 within the enclosure. The syringe 110 can be inserted into the enclosure 120 while being forced distally such that a distal end of the raised rib 111 is pushed into the closed or distal end of the slot 121. This will cause an interference fit between the distal end of the raised rib 111 and the distal end of the slot 121 providing a stop such that the syringe 110 cannot be positioned more distally in the enclosure 120. Such geometric positioning sets the location of the tip of the needle 116 in the parenteral injection system 100 within manufacturing tolerances. Thus, the slot 121 sets the working depth of the needle 116 from a surface of a patient's skin based on the overall length of the needle 116 as connected to the hub 118 and the total distance from the distal end of the slot 121 to the tip of the needle 116.

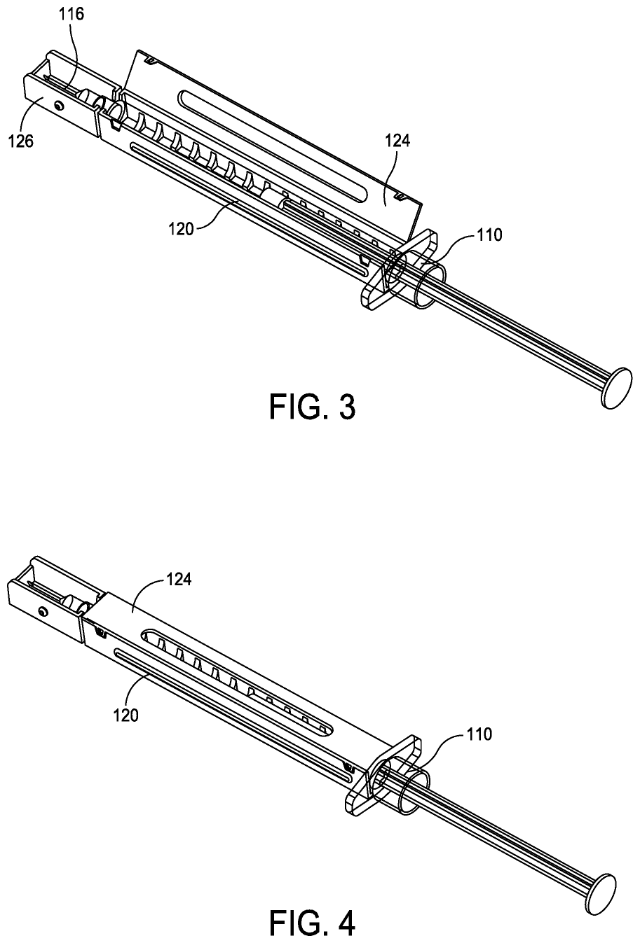

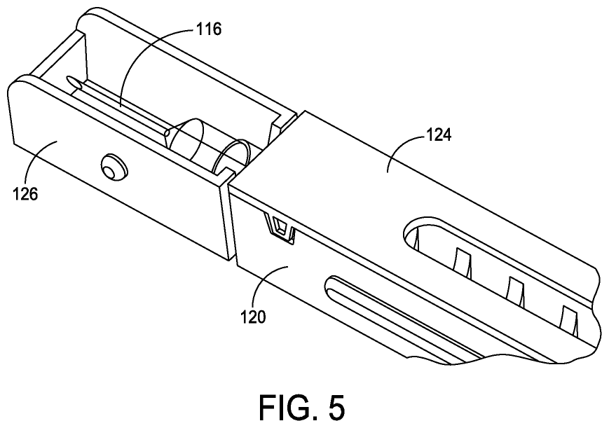

FIG. 3 shows the injector 110 placed within the enclosure 120 and that the enclosure 120 is sized to appropriately fit the injector 110. In this state, the needle 116 is protected within the needle protector 126. FIG. 4 illustrates the injector 110 with the enclosure 120 and the cover 124 closed to secure the injector 110 in the enclosure 120. In this state, the injector 110 and enclosure 120 assembly can be safely stored (optionally in a package) and transported without the risk of the needle 116 being contaminated, damaged, causing sharps errors, or inadvertently poking something or someone. FIG. 5 is a closer view showing the needle 116 within the needle protector 126.

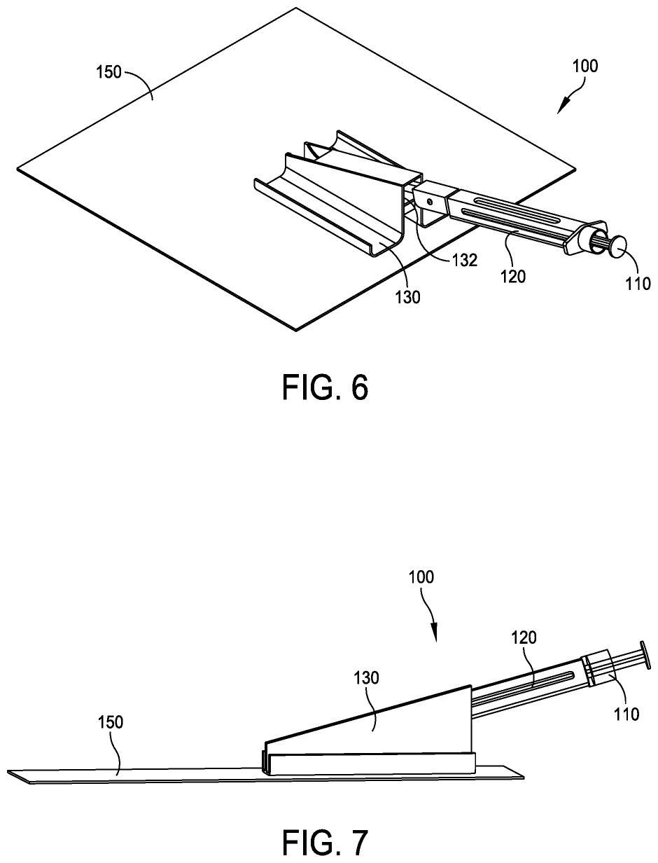

FIGS. 6-10 are used to describe an injection with the parenteral injection system 100. FIG. 6 is a perspective view of the parenteral injection system 100 showing an orientation of the enclosure 120 with injector 110 as it is being located for injection. FIG. 6 shows the sled 130 placed on a patient's skin 150 and the enclosure 120 with injector 110 ready to be fed into a shaft, channel, or sleeve 132 of the sled 130. The angle of the sleeve 132 determines the angle in which the needle 116 approaches and punctures the skin 150. FIG. 7 is a side view of the parenteral injection system 100 with the enclosure 120 with injector 110 partially fed into the sleeve 132.

The sled 130 can include a contact portion that is a rigid planar member, in which the bottom of the contact portion is configured to contact a skin surface of a patient. The right side and the left side of the contact portion can include curved ends that curve upwards towards the top of the sled 130. The bottom of the contact portion can have a smooth surface and curves upwards at the curved ends. The bottom of the contact portion can be pressed such that the patient's skin 150 is taut, providing the needle 116 of the injector 110 with a flat surface for injection. As such, a user does not need to stretch the skin taut with two fingers in a conventional manner. Moreover, the curved ends permit the skin to roll around the curved ends without injuring the skin 150, as opposed to being pressed against sharp edges if for instance, the contact portion is formed having a substantially planar surface and no curved ends.

The sleeve 132 can be a rigid channel defined of guide walls between a first end and a second end of the sleeve. The sleeve 132 can define a substantially rectangular channel in which to fit a rectangular enclosure 120. The first end of the sleeve 132 can include an opening to receive the enclosure 120. The second end of the sleeve 132 can include an aperture that is sized to receive the needle protector 126. The guide walls can be positioned on the right side and left side of the sleeve 132. A portion of the sleeve 132 can be open allowing the user to view a portion of the enclosure 120 and/or the injector 110. In one or more other cases, the sleeve 132 can include guide walls or a singular guide wall that encloses the enclosure 120, such that portions of the injector 110 are not physically exposed while in the sleeve 132. In such cases, the sleeve 132 can be defined from a transparent, semi-opaque, or other like material that allows a user to see the enclosure 120 and/or injector 110 within the sleeve 132.

Although not shown, the sleeve 132 can include features to make it easier for a user to insert and orient the enclosure 120. For example, the first end of the sleeve 132 including the opening to receive the enclosure 120 can be tapered outwardly, ramped, throated, or be wider than the interior of the sleeve. This provides greater flexibility and tolerance for a user to orient the enclosure 120 while it is being initially fed into the sleeve 132. In an aspect, one or more of the sides defining the sleeve 132 can be tapered or angled outwardly to effectively provide a ramp to assist a user to orient and guide the enclosure 120 into the sleeve 132. In an aspect, the sleeve 132 can be shaped or include a keying feature(s) so that the enclosure 120 can be inserted into the sleeve 132 in only one orientation. For example, the sleeve 132 and the enclosure 120 can be shaped (such as a “D” shape or another shape) or otherwise include features such that the enclosure 120 can only be inserted into the sleeve 132 with the open side of the needle protector 126 facing toward the patient's skin.

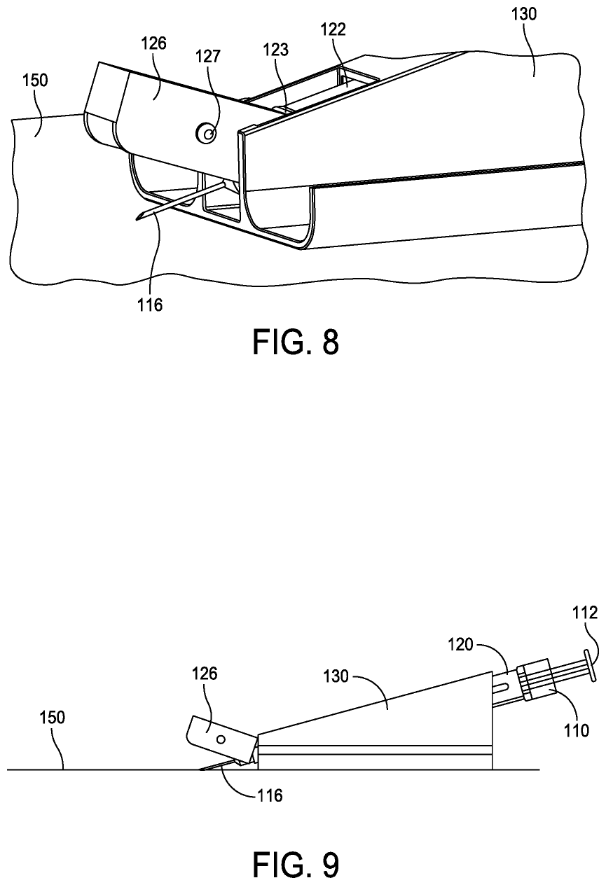

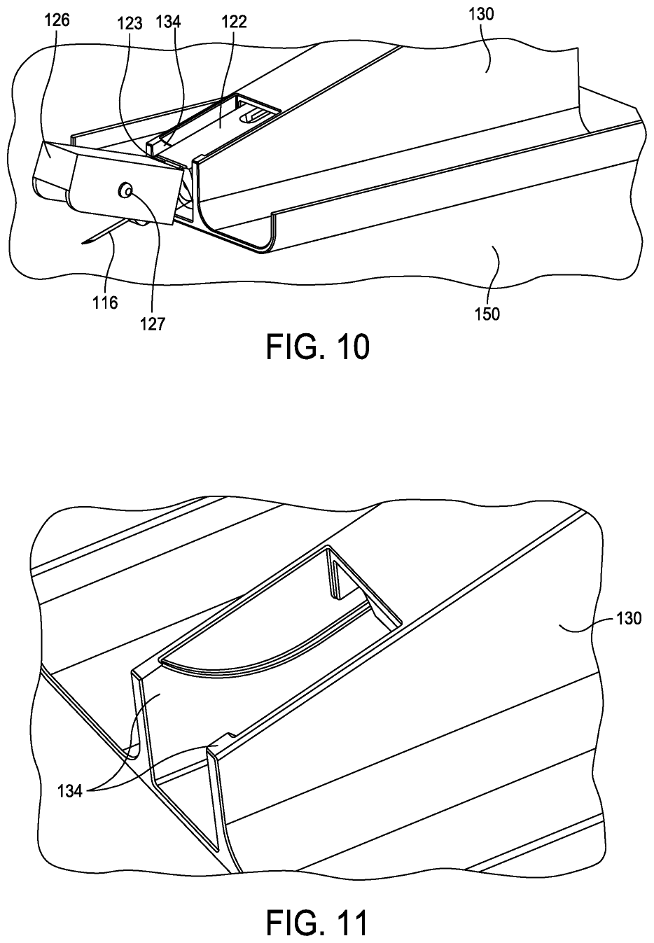

FIGS. 8-10 are different views showing the needle 116 protruding from the enclosure 120 as the needle 116 approaches or punctures the skin 150. FIG. 8 shows that as the enclosure 120 is fed into the sled 130 in an intermediate position, the needle protector 126 is rotated at an angle up and away from the skin 150 with respect to the body 122 of the enclosure 120 to expose the needle 116 and reveal the needle path to the skin 150. The needle protector 126 can be attached to the body 122 of the enclosure 120 via a hinge portion 123 that permits the needle protector 126 to rotate away from the surface with respect to the body 122. Tabs 127, which protrude from opposing sides of the needle protector 126 towards inside walls of the sleeve 132, can ride on curved ramped surfaces of protrusions 134 on the inside walls of the sleeve 132 (best viewed in FIGS. 10 and 11). As the enclosure 120 is forced into the sleeve 132, the tabs 127 contact the ramped surfaces of the protrusions 134, which provides a force to rotate the needle protector 126 up to expose the needle 116. This rotational force on the needle protector 126 is enough to disengage or unsnap the press fit of the detent 129 inside the needle protector 126 from the hub 118 of the injector 110. Once the needle protector 126 has been disengaged from the hub 118, the detent 129 can contact and rest on hub 118 to hold the needle protector 126 in the open position, as shown in FIGS. 8-10.

In an aspect, rather than the ramped surfaces of protrusions 134 forcing up the needle protector 116, the tabs 127 can be situated within a recess, slot, or track defined on each of the inside walls of the sleeve 132. The tracks can be configured in a similar “J” shape as the ramped surfaces of the protrusions 134. An end of the tracks can be open to allow the tabs 127 to exit the sleeve 132, as shown in FIGS. 8-10, or the tracks can be closed at their ends and the tabs 127 repositioned so that the tabs 127 do not exit the sleeve 132, but are retained in their respective tracks.

FIG. 9 is a side view of the parenteral injection system 100 showing that the enclosure 120 has been fed into the sleeve 132 to the point that the needle protector 126 has been rotated to expose the needle 116, and the needle 116 has punctured the skin 150 at the predetermined angle from parallel to the surface of the skin 150 and at the predetermined depth. In an embodiment, the angle is about 11 degrees and a target depth from the surface of a patient's skin is about 1.4 mm, within manufacturing tolerances. FIG. 10 is a perspective view of the parenteral injection system 100 in an injection position. Once the needle 116 punctures the skin 150, a user can administer the medicament within the injector, for example by depressing the plunger 112 and corresponding plunger rod.

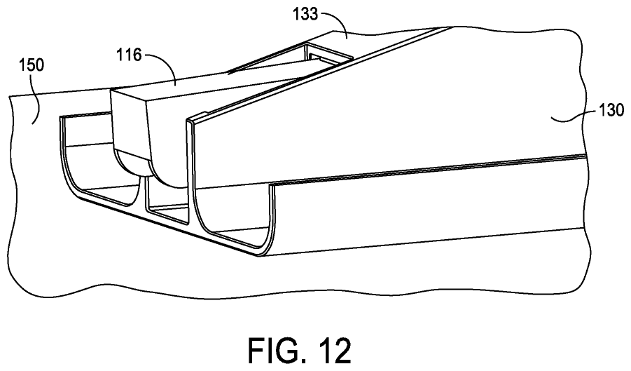

Once the medicament has been injected, the enclosure 120 with the injector 110 can be withdrawn from the sleeve 132. During the action of withdrawing the enclosure 120 from the sleeve 132, the needle protector 126 follows a reverse “J” path from that traveled during insertion of the enclosure 120 into the sleeve 132. As illustrated in FIG. 12, as the enclosure 120 is withdrawn, the needle protector 126 hits an edge of the top wall 133 of the sleeve 132. Continued withdrawal force translates to additional interference force between the needle protector 126 and the top wall 133 forcing the needle protector 126 toward the needle 116. This interference force builds until it overcomes the force necessary for the detent 129 inside the needle protector to reengage or snap back on to the hub 118 of the injector 110. When the enclosure 120 is fully removed from the sleeve 132, the needle protector is in the same protective position as when it was in the transport and storage state shown in FIG. 4.

In the case, where the tabs 127 are located within tracks on the inside walls of the sleeve, during withdrawal of the enclosure 120, the tabs can travel in the reverse direction in the tracks to force the detent 129 of the needle protector 116 to reengage the hub 118.

Upon withdrawal after use, it is expected that the enclosure 120 and injector 110 will be safely discarded into a biological waste dispenser, sharps container, or suitable biohazard equivalent.

In one or more cases, to administer the medicament or fluid to a patient, a user fills the barrel 142 of the injector 110, and subsequently inserts the injector 110 into the enclosure 120 that is fed into the sleeve 132 of the sled 130. The user can use one hand to hold the sled 130 into position on the patient's skin 150 and use the other hand to feed the enclosure 120 into the sleeve 132 of the sled 130. The enclosure 120 with injector can be inserted into the sleeve 132 until the tabs 127 of the needle protector 126 contact the “J” ramped surfaces of the protrusions 134 inside the walls of the sleeve 132. Forcing the enclosure 120 further into the sleeve 132 causes the needle protector 126 to rotate and expose the needle 116. Forcing the enclosure 120 further into the sleeve 132 causes the needle 116 to puncture the skin 150. When the user visually sees that the needle 116 is inserted into the skin 150, the user can administer the medicament or other fluid into the patient, for example by depressing the plunger 112 of the injector 110. To remove the needle 116 from the skin 150, the user can either pull the enclosure 120 with the injector 100 out of the sleeve 132 or remove the system 100 entirely from the surface of the skin 150.

The various embodiments described above are provided by way of illustration only and should not be construed to limit the claims attached hereto. Those skilled in the art will readily recognize various modifications and changes that may be made without following the example embodiments and applications illustrated and described herein, and without departing from the spirit and scope of the following claims.

What is claimed is:

1. A parenteral injection system, comprising:
a sled including a sleeve and configured to be in contact with a patient's skin; and
an enclosure configured to secure an injector having a needle and be fed into the sleeve.

2. The system of claim 1, wherein the enclosure is configured to hold or store the injector that has been occupied with a medicament.

3. The system of claim 1, wherein the injector is a syringe.

4. The system of claim 1, wherein the enclosure in one of a kit of enclosures and is color coded.

5. The system of claim 1, wherein the sled and/or the enclosure glows in the dark.

6. The system of claim 1, wherein the sleeve is arranged to be at a predetermined angle with respect to the patient's skin while the sled is in contact with the patient's skin.

7. The system of claim 6, wherein an angle of the needle and an insertion depth of the needle corresponds to the predetermined angle of the sleeve.

8. The system of claim 1, wherein the enclosure is a substantially cubic box that includes a body, a cover, and a needle protector.

9. The system of claim 8, wherein the cover includes a locking feature to secure the cover to the body when the enclosure is closed.

10. The system of claim 8, wherein the cover includes a clear portion in which to view the injector while inside the enclosure.

11. The system of claim 8, wherein the needle protector is rotatably attached to the body and covers the needle while the injector is secured in the enclosure.

12. The system of claim 1, wherein the body is keyed with the injector such that the needle must be in a predetermined orientation for the injector to be secured in the enclosure.

13. The system of claim 1, wherein the sleeve includes a keying feature such that the enclosure must be in a predetermined orientation to be inserted into the sleeve.

14. A method of parenteral injection, comprising:
securing an injector into an enclosure including a needle protector that covers a needle of the injector and is rotatably attached to the enclosure, the injector including a medicament;
pressing a sled including a sleeve on a surface of a patient's skin;
feeding the enclosure into the sleeve until the needle protector rotates to expose the needle;
puncturing the patient's skin with the needle; and
delivering the medication into the patient's skin.

15. The method of claim 14 further comprising, causing the needle protector to rotate and cover the needle while withdrawing the enclosure from the sleeve.

16. The method of claim 14, wherein an operator presses the sled with one hand and feeds the enclosure, punctures the patient's skin, and delivers the medication with a second hand.

17. The method of claim 14, wherein pressing the sled on the patient's skin makes the patient's skin taut.

18. The method of claim 14, wherein the sleeve includes a keying feature such that the enclosure must be in a predetermined orientation to be inserted into the sleeve.

19. The method of claim 14, wherein the enclosure is keyed with the injector such that the needle must be in a predetermined orientation for the injector to be secured in the enclosure.

20. The method of claim 14, wherein an angle and depth of the needle with respect to the surface of the patient's skin is predetermined by a configuration of the needle within the enclosure and the enclosure within the sleeve.
